(19) United States
(12) Patent Application Publication
(10) Pub. No.: US 2010/0130937 A1
(43) Pub. Date: May 27, 2010

(54) INTRODUCER SHEATH AND METHODS OF MAKING

(75) Inventor: Laveille K. Voss, Belmont, CA (US)

Correspondence Address: WORKMAN NYDEGGER, 1000 EAGLE GATE TOWER,, 60 EAST SOUTH TEMPLE, SALT LAKE CITY, UT, 84111, US

(73) Assignee: ABBOTT VASCULAR INC., Santa Clara, CA (US)

(21) Appl. No.: 12/695,975

(22) Filed: Jan. 28, 2010

Related U.S. Application Data
(63) Continuation-in-part of application No. 11/427,306, filed on Jun. 28, 2006.
(60) Provisional application No. 60/695,602, filed on Jun. 30, 2005.

(51) Int. Cl. A61B 17/34

(52) U.S. Cl. 604/164.03; 604/164.01

(57) ABSTRACT

An introducer sheath and methods of making the introducer sheath are described. The introducer sheath may include a hub portion and a tubular portion. The tubular portion may extend from the hub portion. A lumen may extend between a proximal end and a distal end of the tubular portion. At least a portion of an inner surface and/or outer surface of the tubular member may be irregular.

### CROSS-REFERENCE TO RELATED APPLICATIONS

This application is a continuation-in-part of U.S. patent application Ser. No. 11/427,306, entitled “Introducer Sheath,” filed Jun. 28, 2006, which claims the benefit of U.S. Provisional Patent Application Ser. No. 60/695,602, entitled “Introducer Sheath,” filed Jun. 30, 2005, each of the preceding are incorporated herein by reference in its entirety. This application relates to U.S. patent application Ser. No. 11/427,301, entitled “Modular Introducer and Exchange Sheath,” and filed Jun. 28, 2006, which claims the benefit of U.S. Provisional Patent Application Ser. No. 60/695,464, entitled “Modular Introducer Sheath,” and filed Jun. 30, 2005; U.S. patent application Ser. No. 11/767,947, filed Jun. 25, 2007, and entitled “Expandable Introducer Sheath to Preserve Guidewire Access,” which is a continuation in part of U.S. patent application Ser. No. 11/427,308, filed Jun. 28, 2006, and entitled “Expandable Introducer Sheath;” U.S. patent application Ser. No. ______ filed ______, and entitled “Introducer Sheath and Methods of Making,” (Attorney Docket No. 16497.13.1.1); U.S. patent application Ser. No. ______ filed ______, and entitled “Modular Introducer and Exchange Sheath,” (Attorney Docket No. 16497.13.1.3); U.S. patent application Ser. No. ______ filed ______, and entitled “Expandable Introducer Sheath to Preserve Guidewire Access,” (Attorney Docket No. 16497.14.1.1); and U.S. patent application Ser. No. ______ filed ______, and entitled “Expandable Introducer Sheaths and Methods for Manufacture and Use,” (Attorney Docket No. 16497.14.2), the disclosures of which are hereby incorporated by reference in their entireties.

### BACKGROUND OF THE INVENTION

1. The Field of the Invention

The present invention relates generally to medical devices and methods. More specifically, embodiments of the invention relate to introducer sheaths and methods of making.

2. The Relevant Technology

A wide variety of sheaths have been developed for use in medical procedures. Sheaths are often used, for example, to access a vessel or artery to allow a surgical procedure to be performed. Sheaths are also used for medical procedures that utilize catheters such as, angioplasty or stenting. In practice, the introducer sheath is generally inserted into the patient's vasculature using the modified Seldinger technique. In the Seldinger technique, a needle is first inserted into the vessel and a guidewire then follows through the needle. Next, the needle is removed and a sheath/dilator combination is advanced over the guidewire. The dilator expands the puncture in the vessel to a size suitable to receive the distal end of an introducer sheath. After the distal end of the sheath is disposed within the vessel, the dilator and guidewire are removed, thereby allowing access to the vessel lumen or other body lumen via the inserted introducer sheath.

Conventionally, introducer sheaths are formed of three or more components that require assembly: a sheath portion, a hub, and a hemostasis valve disposed within the hub. A suitable example of such an assembly is shown in U.S. Pat. No. 5,807,350, which shows an introducer sheath having a construction similar to that described above, the entirety of which is hereby incorporated by reference.

Sheaths such as that described above are generally constructed of multiple pieces that must be assembled to form the sheath. Because the sheath is assembled from separate components, it is often difficult to align the lumen of the distal sheath portion with the lumen of the hub. As a result, additional time must be taken during manufacture to ensure alignment thereby leading to increased costs.

In some instances, the hub at the proximal end of the introducer sheath may be overmolded over the elongated sheath portion. While overmolding may produce a stronger sheath, there is the possibility of damaging a portion of the introducer sheath during the overmolding process. In addition to the cost of the overmolding process, the entire introducer sheath would then have to be discarded. There is a therefore a need for a new introducer sheath having lower manufacturing costs.

### BRIEF SUMMARY OF THE INVENTION

These and other limitations may be overcome by embodiments of the present invention, which relates generally to medical devices and methods of use and in particular to introducer sheaths. Embodiments of the invention may provide several designs and methods of manufacture of an improved introducer sheath.

An embodiment of an introducer sheath is described. The introducer sheath may include a hub portion and/or an elongate tubular portion. The elongate tubular portion may extend from the hub portion. The elongate tubular portion may include a lumen that extends from a distal end toward a proximal end of the elongate tubular portion. The lumen may include a plurality of protrusions and/or a plurality of depressions.

In some embodiments, the elongate tubular portion may define a plurality of lumens. Each of the plurality of lumens, in further embodiments, may include a plurality of protrusions and/or a plurality of depressions within each lumen. In still further embodiments, the plurality of protrusions may define a first inner dimension within each lumen and/or the plurality of depressions may define a second inner dimension within each lumen. The first inner dimension may be smaller than the second inner dimension in each lumen.

The protrusions and/or depressions, in some embodiments, may extend from a proximal end to a distal end. In further embodiments, the protrusions and/or depressions may vary in angular orientation with respect to the longitudinal axis between a proximal end and a distal end.

In some embodiments, the plurality of protrusions and the plurality of depressions may extend longitudinally through the lumen. The tubular portion, in further embodiments, may include at least one weakened region and/or at least one stiffened region.

The protrusions and/or the depressions, in some embodiments, may define a friction reducing surface configured to contact an outer surface of a medical device.

A further embodiment of an introducer sheath is described. The introducer sheath may include a hub portion and/or an elongate tubular portion. The elongate tubular portion may extend from the hub portion. The elongate tubular portion may have a first lumen and/or a second lumen. The first lumen may include a plurality of first protrusions and/or a plurality of first depressions. The second lumen may include a plurality of second protrusions and/or a plurality of second depressions.

In some embodiments, the plurality of first protrusions and/or the plurality of first depressions may differ from the plurality of second protrusions and/or the plurality of second depressions. The plurality of protrusions and/or the plurality of depressions of the first inner surface, in further embodiments, are parallel from the proximal end toward the distal end.

A still further embodiment of an introducer sheath is described. The introducer sheath may include a hub portion and/or an elongate tubular portion. The elongate tubular portion may extend from the hub portion. The elongate tubular portion may have a lumen. The lumen including a plurality of protrusions and/or a plurality of depressions. The plurality of protrusions may define a first inner dimension. The plurality of depressions may define a second inner dimension. The first inner dimension may be smaller than the second inner dimension.

In some embodiments, the first inner dimension may be about fifty percent smaller than the second inner dimension. The first inner dimension may be from about thirty percent to about sixty percent smaller than the second inner dimension. The first inner dimension may be from about twenty percent to about seventy percent smaller than the second inner dimension.

A yet further embodiment of an introducer sheath is described. The introducer sheath may include a hub portion and/or an elongate tubular portion. The elongate tubular portion may extend from the hub portion. The elongate tubular portion may have a lumen. The lumen may include a plurality of protrusions and/or a plurality of depressions. The plurality of protrusions may define a first wall thickness. The plurality of depressions may define a second wall thickness. The first wall thickness may be larger than the second wall thickness.

In some embodiments, the first wall thickness is about fifty percent larger than the second wall thickness. The first wall thickness may be from about thirty percent to about sixty percent smaller than the second wall thickness. The first wall thickness may be from about twenty percent to about seventy percent smaller than the second wall thickness. In some embodiments, the tubular portion may include PTFE or FEP.

An embodiment of a method for performing a medical procedure is described. The method may include introducing a sheath into a lumen of a patient. The sheath may have a first unexpanded dimension and/or an irregular wall surface. A first medical device may be inserted into the lumen through the sheath to perform a medical procedure. The first medical device may have an outer dimension. At least a portion of a tubular member of the sheath may expand to a second expanded dimension to accommodate the outer dimension of the first medical device.

In some embodiments, a second medical device may be inserted through the sheath. Inserting a second medical device through the sheath may include introducing a vessel closure device through the sheath and/or closing the lumen of the patient with the vessel closure device. The sheath, in further embodiments, may include a tubular portion extending from the hub portion. The tubular portion may include at least one portion deformable to increase a cross sectional area of the tubular portion. In still further embodiments, the at least one portion may be splittable to increase a cross sectional area of the tubular portion.

The sheaths disclosed herein can be used with various medical devices. In one configuration, the sheath can be used in combination with a vessel closure device, such as those shown in U.S. Pat. No. 6,197,042 and pending U.S. patent application Ser. No. 10/638,115 filed Aug. 8, 2003 entitled “Clip Applier and Methods,” each of these are assigned to a common owner and herein incorporated by reference in their entireties.

Additional features and advantages of the invention will be set forth in the description which follows, and in part will be obvious from the description, or may be learned by the practice of the invention. The features and advantages of the invention may be realized and obtained by means of the instruments and combinations particularly pointed out in the appended claims.

### BRIEF DESCRIPTION OF THE DRAWINGS

In order that the manner in which the above-recited and other advantages and features of the invention are obtained, a more particular description of the invention briefly described above will be rendered by reference to specific embodiments thereof which are illustrated in the appended drawings. Understanding that these drawings depict only typical embodiments of the invention and are not therefore to be considered limiting of its scope, the invention will be described and explained with additional specificity and detail through the use of the accompanying drawings in which:

FIG. 1A is a plan view of an exemplary embodiment of an introducer sheath in accordance with the present invention;

FIG. 1B illustrates a cross-sectional view of the sheath in FIG. 1A and illustrates a valve disposed in the sheath's hub and an alignment member;

FIG. 1C is a cross-sectional view taken along line 1C-1C of the sheath of FIG. 1A in accordance with the present invention;

FIG. 2A illustrates a cross-sectional view of another sheath in accordance with the present invention;

FIG. 2B is cross-sectional view of an alternative embodiment of the sheath of FIG. 2A illustrating the geometric features formed within wall of the sheath in accordance with the present invention;

FIG. 2C is a cross-section view of a portion of an another alternative embodiment of the sheath of FIG. 2A in accordance with the present invention;

FIG. 3A is a plan view of an alternative embodiment of a sheath in accordance with the present invention;

FIG. 3B is a cross-sectional view of the sheath of FIG. 3A taken along line 3A-3A in accordance with the present invention; and

FIG. 3C illustrates a cross-sectional view of an alternative embodiment of a sheath in accordance with the present invention.

FIGS. 4A-8B illustrate cross-sectional views of various embodiments of a sheath in accordance with the present invention.

### DETAILED DESCRIPTION

Due to the general nature of an elongate tubular member, the longer the member, the more column strength and/or other factors may be considered. Buckling and/or kinking during a procedure using an introducer sheath may hinder the procedure. The types of materials in some embodiments of introducer sheaths, may also affect the column strength and/or kink resistance of the sheath. For example, kink occasions have been observed in some instances where a polytetrafluoroethylene (“PTFE”) introducer sheath is used for a prolonged procedure.

An irregular wall design may provide at least one of the following features. For example, an irregular wall design may provide increased column strength and/or kink resistance while maintaining an outer diameter sufficient to perform various medical procedures. In another example, an irregular wall design may minimize friction between the inner surface and/or outer surface of the introducer sheath and a medical device to be inserted into the introducer sheath and/or tissue near an insertion site, respectively.

An irregular wall design may include variations in wall thickness about a circumference (i.e. perimeter) of at least a portion of the introducer sheath. For instance, a plurality of protrusions and/or depressions about a portion of an inner surface and/or outer surface of an introducer sheath may be provided. For example, a typical introducer sheath may have a generally uniform inner diameter and/or outer diameter. While an introducer sheath with an irregular wall design may have at least some portions of the inner surface and/or outer surface that may be nonuniform.

An introducer sheath in accordance with the present invention is described herein as having portions or members, though it shall be understood that the introducer sheath as described herein may be formed as a unitary member such that the portions or members are portions or members of a unitary device. Embodiments of the introducer sheath are depicted in the drawings, which are not necessarily to scale and are not intended to limit the scope of the invention. It will be understood that the benefits of the present invention are not limited to application with an introducer sheath. Rather, other medical devices may be modified based upon the teaching contained herein such that they to can provide the identified functionality.

The introducer sheath may be formed, by way of example, using a co-extrusion process or an injection molding process or other method that results in a sheath formed as a unitary member. The process by which an introducer sheath is formed may include the use of one or more materials. The materials can be used simultaneously, or at different stages of the manufacturing process.

Typically, the materials used to form the introducer sheath include medical grade synthetics or plastics. Exemplary materials may include, but are not limited to, flexible PVC, polyurethane, silicone, liner low-density polyethylene (“LLDPE”), polyethylene, high density polyethylene, (“DHPE”), polyethylene-lined ethylvinyl acetate (“PE-EVA”), polypropylene, latex, thermoplastic rubber, (“PTFE”), fluorinated ethylene propylene (“FEP”), other materials, or combinations thereof. In some embodiments, the materials are configured to have chemical resistance, crack resistance, no toxicity, Food and Drug Administration (“FDA”) compliance, non-electrical conductivity, dimensional stability, be sterilized by ethylene oxide, gamma radiation, autoclave, UV light, ozone, other configurations, or combinations thereof.

In addition, the selection of materials for a particular sheath can depend on a variety of factors that may include, but are not limited to, a particular stiffness and/or flexibility of the sheath or any portion of the sheath, including the desired column stiffness and strength to enable insertion of the sheath, a particular shear or split strength for the sheath or any portion of the sheath, the ability to resist kinking, and the like. For example, the material used for the tubular portion of the introducer sheath may be selected based on shear strength or how easily it can be split. Further, certain features of the sheath may be formed to enhance certain characteristics. For example, a strain relief portion may be formed to resist kinking while the elongated tubular portion may be formed to facilitate splitting.

When more than one material is used to form the sheath or to form specific portions of the introducer sheath, the materials may be selected, in addition to the factors identified herein, on a bond strength between the materials and/or on the elasticity of a particular material. The bond strength, for example, may have an impact on the splitability of the sheath or of a portion of the sheath. The bond strength may also affect the ability of the sheath to expand without splitting.

As described above, the materials of a sheath may be selected based on a splitting or shear property of the materials. One reason for this characteristic or property relates to use of the sheath in medical procedures. For example, when the sheath is used in conjunction with a medical device during a medical procedure, it may be desirable for the introducer sheath to split or shear during insertion or retrieval of the medical device. This may occur, for example, when a vessel is closed with a vessel closure device. The vessel closure device can be used to attach a clip that effectively seals or closes the entry to the body lumen. As the entry or access to the body lumen is closed, the vessel closure device can apply a force that causes the sheath to split. Embodiments of the invention thus contemplate embodiments of the sheath or of portions of the introducer sheath that facilitate splitting at the appropriate time. Further, embodiments of the sheath contemplate structural features that relate to the ease with which a sheath splits without otherwise impacting the use of the sheath.

In accordance with one embodiment of the present invention, an introducer sheath may include a hub member or hub portion having a proximal end and a distal end. The proximal end of the hub portion may include and/or be configured to receive a flexible valve member therein. The sheath may further include an elongated tubular portion generally extending from the distal portion of the hub member. The elongated tubular portion is generally centered with an axis of the hub member and the lumen of the tubular portion is aligned with a lumen of the hub portion because the sheath is formed as a single integrated unit in some embodiments. Alternatively, the lumen of the tubular portion can be aligned with a lumen of the hub portion, whether or not axially aligned. The aligning of the lumens can occur during manufacture, such as when the hub portion and the sheath are formed as a single integrated unit.

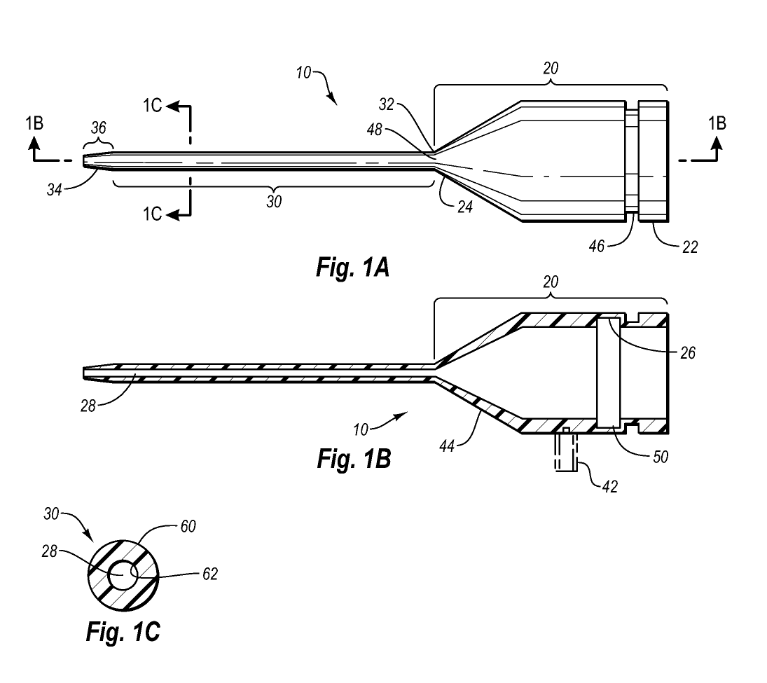

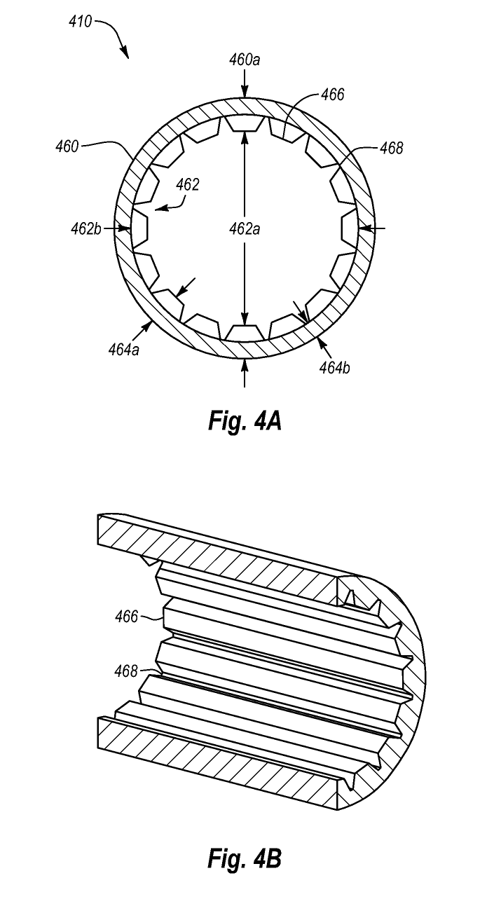

Referring now to FIG. 1A, there is shown an exemplary embodiment of an introducer sheath 10. The introducer sheath 10 can include a hub portion 20 having a proximal end 22 and a distal end 24 and a tubular portion 30 having a proximal end 32 and a distal end 34. The cross section of the hub portion 20 may be generally cylindrical in nature, although other configurations are contemplated. Exemplary configurations or shapes may include, by way of example, oval, polygonal, elliptical, or other cross-section that can be usable for a medical device that is insertable into a body lumen. FIGS. 4A-8B provide examples of various cross-sectional portions.

The elongate tubular portion 30 may extend from the distal end 24 of the hub portion 20. Because the sheath 10 can be formed as a unitary member, the proximal end 32 of the tubular portion 30 can be integrally formed with the distal end 24 of the hub portion 20. Because the sheath 10 can be formed as a unitary member, the hub portion 20 may effectively transition to the tubular portion 30. Because the transition between the hub portion 20 and the tubular portion 30 may introduce a natural flex point, embodiments of the invention may include a strain relief portion 48, which may provide a more effective transition of the tubular portion 30 of the sheath 10 to the hub portion 20. The strain relief portion 48 may be formed at the transition between the hub portion 20 and the tubular portion 30. More particularly, the strain relief portion 48 may be disposed adjacent the distal end portion of the hub portion 20 and adjacent the proximal end 32 of the elongate tubular portion 30.

The strain relief portion 48 may also be configured to provide additional support to at least the proximal end 32 of the elongate tubular portion 30 to reduce and/or prevent kinking at the transition zone of the proximal end 32 of the elongated portion 30 and the distal end 24 of the hub portion 20. In one embodiment, the strain relief portion 48 may be formed by gradually increasing a thickness of tubular portion 30 as the tubular portion 30 of the sheath 10 transitions to the hub portion 20 of the sheath. Alternatively, the strain relief portion 48 may be formed using other structures or formations that provide, for example, support or kink resistance to the transition from the tubular portion 30 to the hub portion 20. For instance, the strain relief portion 48 may include webs, extensions, other internal or external structures, or combinations thereof to increase the strength and/or stiffness of the introducer sheath 10 at the hub portion/tubular portion transition.

With continued reference to FIG. 1A, the distal end 34 of the tubular portion 30 may include a tapered portion 36 that may facilitate entry of the introducer sheath 10, for example, into patient's vasculature or other body lumen. The tapered portion 36 may be formed after the initial forming process of the introducer sheath 10 or be formed as part of the initial forming process. For example, the tapered portion 36 may be formed as part of the extrusion or injection molding processes. Alternatively, the tapered portion 36 may be formed by heat forming, grinding, milling, laser treatment, etching, other methods, or combinations thereof that may produce a thinner wall thickness.

FIG. 1B further illustrates a cross-sectional view of the sheath 10 along the line 1B. As shown, a lumen 28 extends from a proximal end 22 of the hub portion 20 to the distal end 34 of the tubular portion 30. The lumen 28 may be generally uniform in cross-section over all or a portion of its length between the proximal end 22 of the hub portion 20 and the distal end 34 of the tubular portion 30. In the illustrated configuration, the lumen 28 has a generally uniform cross-section along its length along the tubular portion 30, while having a generally uniform cross-section portion and a changing cross-section portion along the length of the hub portion 20. It will be understood, however, that other cross-sectional configurations are possible so long as they can accommodate a medical device or instrument inserted therein. For example at least one of the cross-sections illustrated in FIGS. 4A-8B may be implemented.

With continued reference to FIG. 1B, the proximal end 22 of the hub portion 20, within the lumen 28 and defined by the inner wall or surface 52 forming the lumen 28, may include a feature, such as a receiving feature 26, therein, which may be configured to receive a flexible valve member 50. The valve member 50 may be inserted after the sheath 10 is formed. For instance, the receiving feature 26, such as a groove or channel, can receive the valve member 50 and retain the same within the hub portion 20. In another example, a retaining cap (not shown) may be disposed adjacent to or within the proximal end of the hub portion 20 may aid the receiving feature 26 to retain the flexible valve member 50 within the hub portion 20. In a further example, the valve member 50 may be integrally formed with the hub portion 20 during the molding process of the sheath 10 and as such the hub portion 20 need not include the receiving feature 26.

The cooperation between the receiving feature 26, optional the retaining cap, and/or the valve member 50 may provide a sealed hub portion 20. Stated another way, the valve member 50 may be self sealing once inserted or formed in the hub portion 20 to limit fluid escaping from the body lumen.

The valve member 50 may be one of a variety of different seals, including being self-sealing once it is inserted into the hub portion 20. The valve member 50, for example, may have an elastomeric body, such as silicone rubber or other material as described above, with at least one slit and/or other collapsible opening formed therein to allow selective insertion and removal of medical instruments, such as guidewires, catheters, and other such devices. The collapsible openings or other portions of the valve member 50 may provide a fluid tight seal with or against the medical instrument. Thus, leakage of blood other bodily fluids, and/or fluids such as unwanted air may be inhibited and/or prevented from entering the body. Examples of such flexible membranes or valve members, which can be utilized with the present invention, are shown in U.S. Pat. Nos. 4,798,594, 5,176,652, and 5,453,095 the entireties of which are herein incorporated by reference.

With continued reference to FIG. 1B, illustrated is a port member 42 that may be formed on the outer surface or outer wall 44 of the hub portion 20. The port member 42 may function as a fluid port for the sheath 10. Thus, any fluid, such as saline or blood or medication for example, may be added and/or withdrawn through the port member 42. The port member 42 may be configured to align or position any device or instrument (e.g., a vessel closure device, a catheter) used in conjunction with the sheath 10. The port member 42 may be shaped to interact with an alignment mechanism on a medical device and optionally create a fluid sealed connection. One exemplary type of port member may include a member having a luer lock configuration. It will be understood that other types of ports can perform the desired function.

A retention recess or ring 46, as shown in FIG. 1A may be formed on the outer surface or wall 44 of the hub portion 20. The recess or ring 46 may be used to secure a cap (not shown) to the sheath 10. The recess or ring 46 may have various configurations to perform the identified and desired function. For instance, although the walls forming the recess or ring 46 are illustrated as being generally parallel, the recess or ring 46 may have tapered wall, curved wall, combinations of generally parallel, tapered, or curved walls, or generally any other configuration that would allow a cap to be secured thereto or for the recess.

It is contemplated that the wall thickness along the length of the elongate tubular portion 30 can be varied to vary mechanical properties of the sheath (e.g., kink resistance, stiffness, flexibility and the like). Further, the thickness of the strain relief 40 (which can vary across the transition between the tubular portion 30 and the hub portion 20), the thickness of the hub portion 20, the diameter of the lumen of the tubular portion 30 and of the lumen of the hub portion 20 can also be varied or specifically selected.

These dimensions of the sheath 10 are often controlled and determined during the manufacturing process. In an injection molding process, for example, the sheath 10 may be formed using a mold. The mold can be machined or configured based on the desired dimensions and/or configurations of the sheath 10 as described herein. After the mold (which may include more than one part) is formed, the injection molding process can begin by melting a suitable material, such as one described above, and then injecting the melted material into the mold, often under pressure. The mold used in the injection molding process is typically formed such that the molded introducer sheath can be removed after it has cooled and such that the resulting introducer sheath has the desired dimensions and characteristics described herein. As a result, the molded sheath 10 can be a unitary member and may not be assembled from separately formed parts.

Benefits of forming the introducer sheath 10 as a unitary member may include reduced costs, more accurate parts (i.e. dimension control) due to lack of assembly, as well as the ability to balance mechanical properties across the entire sheath 10. For example, the thickness of the walls of the hub portion, the tubular portion, the strain relief, the tapered portion, other portions, or combinations thereof can be controlled and/or varied as desired.

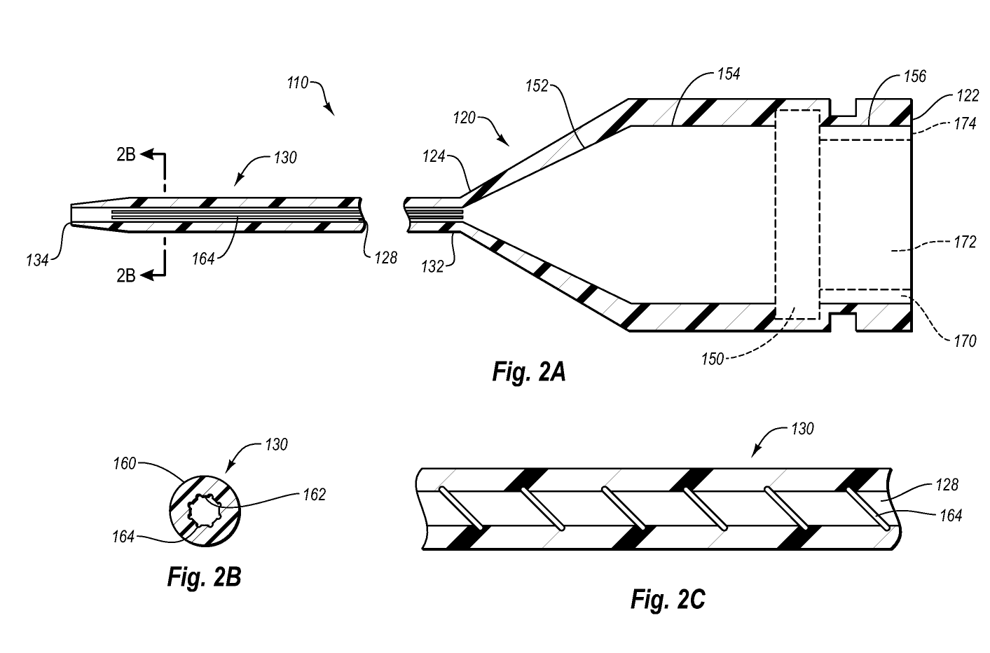

Referring now to FIG. 1C, there is shown a cross-sectional view of the sheath 10 in accordance with the present invention along the line 1C-1C of FIG. 1A. In particular, FIG. 2 illustrates a cross-sectional view of the elongate tubular portion 30 of the sheath 10. The elongate tubular portion 30 can include an outer wall 60 and an inner wall 62 thereby defining a wall thickness. Additionally, the lumen 28 may extend along the length of the tubular portion 30. The width or diameter of the lumen 28 may vary and/or may depend on the intended use of the sheath 10. Because the hub portion 20 and the tubular portion 30 may be integrally formed, the lumen 28 may be axially aligned along its length. Stated another way, the axis of the portion of the lumen 28 within the tubular portion 30 can be aligned with the axis of the portion of the lumen 28 within the hub portion 20.

Generally, the outer wall, whether defined by the outer wall 60 of the tubular portion 30 or the outer wall 44 of the hub portion 20, defines the outer surface or wall of the sheath 10. Similarly, the inner wall, whether defined by the inner wall 62 of the tubular portion 30 or the inner wall 52 of the hub portion 20, defines the inner surface or wall and lumen 28 of the sheath 10.

As mentioned above, although the cross-sectional view of the tubular portion 30 is cylindrical in nature, other cross-sectional shapes (such as those shown in FIGS. 4A-8B) are within the scope of the invention. Further, the lumen 28 may also have an alternative cross-sectional shape other than generally circular. In one example, the cross-sectional shape of the tubular portion 30 and/or the lumen 28 can be determined by the mold used in an injection molding process. Further, the cross-sectional configuration of the lumen 28 need not be the same as that of the cross-section configuration of the tubular portion 30 as defined by the outer wall of the tubular portion 30, and more generally the sheath 10.

Referring now to FIG. 2A there is shown an exemplary embodiment of an alternative introducer sheath in accordance with the present invention. Much of the description related to the sheath 10 may also apply to the present embodiment of the sheath 110, and vice versa. The alternative embodiment of the sheath will herein be described as having portions similar to that as described above.

As shown in FIG. 2A, the sheath 110 can include a hub portion 120 having a proximal end 122 and a distal end 124, and a tubular portion 130 having a proximal end 132 and a distal end 134. Extending from the proximal end 122 to the distal end 134 is a lumen 128. Generally, the configuration of the lumen 128 and the inner wall or surface forming the lumen 128 may be different from that described with respect to lumen 28 (FIG. 1B). A portion of the lumen 128 in the hub portion 120, or the inner wall or surface 152 can have a stepped configuration. The stepped configuration can include a first portion 154 having a first inner diameter and a second portion 156 having a second diameter larger than the first diameter. This stepped configuration, or the transition between the first portion 154 and the second portion 156 provides or functions as a stop for an inserted valve member 150.

The valve member 150 can be secure within the lumen 128 through a friction or interference fit with the inner surface or wall 152 of the hub portion 120. Alternatively, or in addition to the friction or interference fit, the valve member 150 may be mounted within the lumen 128 through adhesives, thermal or chemical bond, mechanical coupling, such as, but not limited to, the use of a groove or recess in the inner surface or wall 152, or other technique used to mount two components together. In one configuration, a retaining cap 170, having a lumen 172 that may receive a medical device or instrument to be inserted through the valve member 150 and/or the lumen 128 may secure the valve member 150. The proximal end 174 of the retaining cap 170 may align with, overlap, or be recessed relative to the proximal end 122 depending upon the particular configuration of the end cap 170.

With reference to FIGS. 2A and 2B, the elongated tubular portion 130 includes an outer surface or wall 160 and an inner surface or wall 162. Formed in the inner wall 162 may be at least one longitudinal groove 164, and more generally a geometric pattern of grooves, channels, recesses, depressions, or other structures, that may extend along an axis parallel to axis extending through the center of the sheath, and may be centered within the lumen 128. With one or more longitudinal grooves 164, the longitudinal grooves 164 can be formed in various patterns and/or orientations to provide different characteristics to the tubular portion 130. It is contemplated that additional styles and types of patterns may be utilized in accordance with the present invention. For example, one or more longitudinal grooves 164 may form a sinusoidal pattern disposed about the inner radius of the elongate tubular portion 130. Alternatively, the one or more longitudinal grooves 164 may be configured to run along a different axis than one parallel to an axis extending along the center of the sheath 10. For example, the one or more longitudinal grooves 164 may be formed as one or more spirals as illustrated in FIG. 2C. The one or more longitudinal grooves 164 may also only extend partially along the length of the tubular portion 130. In another embodiment, the one or more longitudinal grooves 164 may extend beyond the tubular portion 130 and into the hub portion 120 (FIG. 1A). In another example, the one or more longitudinal grooves 164 may not extend into the tapered portion of the tubular portion 130.

Generally, it should be understood that the above described configuration of the at least one groove 164 should be considered exemplary and not limiting in any manner. It is contemplated that additional styles and types of patterns may be utilized in accordance with the present invention. For instance, one configuration of the longitudinal grooves 164 can provide increased column stiffness, while another configuration can provide kink resistance and/or resistance to torsional loads. Further, it should be understood that the inner wall 162 could have patterns or configurations of structures other than grooves to achieve desired configurations. For instance, and not by way of limitation, other dents, extensions, channels, recesses, or other structural formations can be created upon or in the inner wall 162.

The formation of the geometric pattern of the plurality of grooves 164, for example, can be formed by machining a corresponding feature in the mold and subsequently using the mold during compression molding, injection molding, blow molding, rotational molding, other molding and/or fabrication processes, or combinations thereof. As a result, the geometric pattern may be automatically formed during the manufacturing process and additional steps or acts may be unnecessary to form the geometric pattern on the inner wall 162.

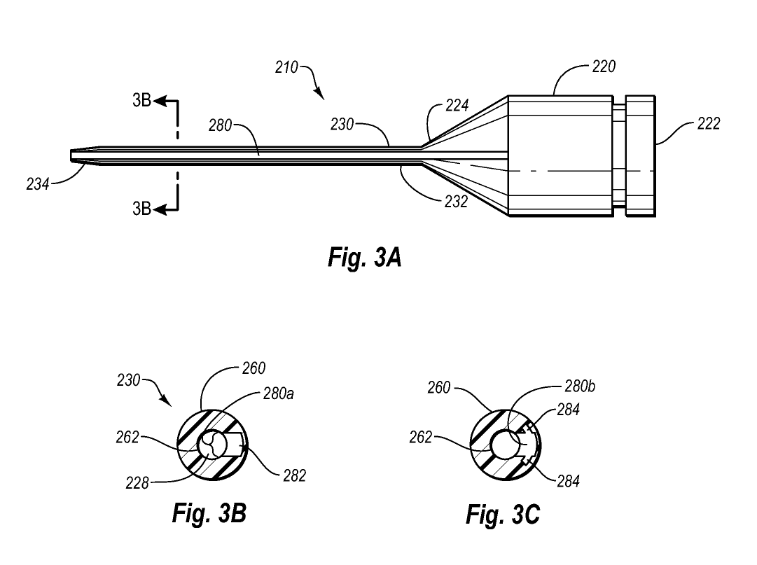

Referring now to FIG. 3A there is shown an exemplary embodiment of an alternative introducer sheath in accordance with the present invention. Much of the description related to sheath 10 and sheath 110 may also apply to the embodiment of the sheath 210, and vice versa. The alternative embodiment of the sheath will herein be described as having portions similar to that as described above.

As shown in FIG. 3A, the sheath 210 may include a hub portion 220 having a proximal end 222 and a distal end 224. The sheath 210 includes a composite elongate tubular portion 230 extending from the distal end 224 of the hub portion 220. In this example, the elongated portion 230 may be generally tubular in construction and may include a proximal end 232 and a distal end 234. As described herein, the cross-sectional shape of both the elongated portion 230 and the hub portion 220 can be any shape, such as by way of example, circular, elliptical, square, polygonal, and the like. In this example, however, the tubular portion may be composite and can be formed from more than one material.

The sheath 210 may additionally include a feature formed within the hub portion 220 that may be configured to receive a flexible valve member (such as the valve member 50 in FIG. 1B or valve member 150 in FIG. 2A). The flexible valve member may be integrally formed into the hub portion during the molding process of the sheath 210 or may be held within the hub portion 220 using the techniques or methods described herein. Alternatively, the hub portion 220 of the sheath 210 may be molded to provide the elements needed to hold the valve member in place after insertion. The receiving feature 26 (FIG. 1B) or the stepped configuration illustrated in FIG. 2A are examples of features that may retain the valve member after insertion into the hub portion 220.

Turning now to the tubular portion 230, and with reference to FIGS. 3A and 3B, at least one groove 280, protrusion, depression, or combinations thereof may be disposed within at least a portion of the tubular portion 230, with one being shown in the illustrated configuration. This groove 280 may receive an insert 282 to provide certain characteristics and properties to the tubular portion 230. For instance, the insert 282 may provide structural stiffness and/or kink resistance to the tubular portion 230 and/or the introducer sheath 210. The groove 280 may extend between (i) the outer surface or wall 260 and the inner surface or wall 262, (ii) the outer surface or wall 260 toward the inner surface or wall 262, (iii) the inner surface or wall 262 toward the outer surface or wall 260, or combinations thereof.

As shown in FIGS. 3A and 3B, the groove 280 and/or the insert 282 may extend from the tubular portion 230 to the hub portion 220. Generally, the groove 280 and/or the insert 282 may extend from a portion of the tubular portion 230 to a portion of the hub portion 220. Alternatively, the groove 280 and/or the insert 282 may be formed only in the tubular portion 230, only in the hub portion 220, or in a portion of the hub portion 220 and/or the tubular portion 230. In other embodiments, one or more grooves 280 and/or inserts 282 may be formed in the sheath 210. Although reference is made to a groove, herein other geometric patterns or configurations of channels, recess, holes, other structures, or combinations thereof formed in the sheath can be used. For example, irregular wall patterns, such as those shown in FIGS. 4A-8B, may be used. Further, a line or other geometric pattern scored or formed in the sheath, with or without the inclusion of the insert can function in a similar manner to the groove and insert as described herein.

With continued reference to FIGS. 3A and 3B, the insert 282 may be formed in the groove 280 in a variety of manners. In one configuration, the groove 280 may be formed as part of the initial molding process. For instance, the sheath 210 may undergo a first injection molding process where the hub portion 220 and elongated portion 230 may be formed as a single unitary unit, with the groove 280 being formed at that time. The mold used to form the sheath 210 may then be adapted, such as by removing the portion of the mold that was responsible for the groove 280, and a second injection molding process may then be performed to inject a second material into the groove 280 to form the insert 282. The insert 282 may effectively bond to the material defining the groove 280 resulting in the sheath, the sheath being a unitary member. One example of a molding technique that can be used to perform the above-described process is an over-molding injection molding process.

It is also contemplated that the first and second injection molding processes can be conducted simultaneously or within a time period of each other, for instance by way of an over-molding injection molding process or a 2-shot injection molding process. In one configuration, a mold can be manufactured and placed into an injection molding machine, wherein the first molding process can form the sheath including the groove 280 shown in FIG. 3A and a second molding process would form the completed sheath by filling the groove 280 with a second material to form the insert 282, resulting in the configuration of FIG. 3B. Thus, the tubular portion 230 can be a composite. The process times can be controlled depending upon the materials to be molded and the desired mechanical properties.

With reference to FIG. 3B, a cross-sectional view of the elongated portion 230 taken about line 3B-3B of FIG. 3A is illustrated. The cross-sectional view of FIG. 3B illustrates the tubular portion 230 after the groove 280 has been formed and filled with a second material, which forms the insert 282. As shown in FIG. 3B, the elongate tubular portion 230 has an outer wall 260 and an inner wall 262 thereby defining a lumen 228 as well as a wall thickness. The insert 282 is shown disposed in groove 280a thereby forming a continuous generally tubular cross-section. In one configuration, the inner wall or surface 262 of the elongated portion 230 typically remains smooth after the second material is injected into the groove 280a to form the insert 282. Alternatively, the inner surface 262 of the elongated portion 230 may have one or more variations, at least one of which may be defined by the insert 282 within the groove 280a. For instance, during the process of applying or depositing the second material the mold defining the boundaries for the second material 282 may include the desired pattern of the portion of the inner wall or surface 262 associated with the insert 282.

As described herein, the second material, as well as the first material, may be chosen based upon desired mechanical properties for the sheath 210. For example, it may be desirable to produce an elongated portion 230 that is may split along a portion of the interface between the first and second materials or through the second material in response to an adequate applied force, including relatively small applied forces. In this case, the bond between the first material and the second material can be adjusted through the manufacturing process. As previously stated, the first and second materials may be selected according to the bond between the first material and the second material and on the splitability of the first and/or second materials. For example, the thickness of the first material at the interface with the second material can be less than the thickness of the first material at other locations. This, combined with a second material that fills the groove 280a to form the insert 282 and may have less strength than the first material, may provide a sheath that has particular properties. For example, the tubular portion 230 may be more likely to split along the groove 280a or along any other geometric pattern formed on the inner wall of the tubular portion 230, whether or not filled with a second material or the insert 282. In instances where the geometric pattern such as the groove 280a is filled with a second material to form the insert 282, a bond may be formed automatically during the molding process. Alternatively, thermal bonding, chemical bonding, or other techniques can be used to facilitate bonding between the similar or dissimilar medical grade materials forming the insert 282 and the remainder of the sheath 210.

As illustrated above, mechanical properties of the tubular portion may be adjusted by forming the elongate tubular portion 230 as a composite member. For example, if it is desirable to produce a sheath that is splittable during use, the second material and/or the insert 282 may be weaker than the first material, thereby forming a joint wherein the sheath may be easily split by an applied force. Alternatively, the second material and/or insert 282 can be utilized to stiffen or weaken the overall tubular portion 230. This can be used to prevent kinking, and the like. Alternatively, the second material and/or insert 282 can be used to stiffen or weaken the overall tubular portion 230 and/or assist in splitting the sheath during use. For example, the second material and/or insert 282 may provide stiffness and/or cause the tubular portion 230 to split at the groove and/or other geometric pattern in response to an applied force, such as the withdrawal of a medical device like a vessel closure device.

Although the alternative embodiment has been described with respect to specific geometries as well as construction methods this should not be considered limiting in any manner. For example, it is contemplated that the groove 280 may be formed having many different geometric shapes, patterns, lengths, or combinations thereof. Additionally, the groove may include a geometric feature formed along the length thereof, wherein the second material and/or insert 282 may at least partially fill into this feature, thereby interlocking the two materials together.

FIG. 3C, for example, illustrates another configuration of the interface between a first material and a second material or between the groove and an insert. In particular, the groove 280b may include sub-grooves 284 that may extend outwardly from the main portion of the groove 280b. These sub-grooves 284 may receive and/or be filled with the second material that may form the insert 282 during the injection molding process and/or provide a mechanical connection and/or coupling between the two materials and/or between the groove 280b and/or the insert 282. As such, the sub-grooves 284, together with the insert 282 and/or second material deposited therein, may function as interlocking features that may mechanically tie the portions of the tubular portion 230 together. By so doing, the two portions of the tubular portion 230 may be mounted and/or coupled together through both the bonding of the two materials and/or the mechanical coupling of the interlocking features formed in the groove 280b and/or the insert 282.

It will be understood that in another configuration, the insert 282 may be formed separately from the remainder of the sheath 210. The insert 282 may then be mounted and/or coupled to the groove 280b during subsequent processing. For instance, the insert 282 can be mounted and/or coupled to the groove 280b using adhesives, thermal or chemical bonding, other techniques, or combinations thereof to mount and/or couple similar and/or dissimilar medical grade materials. Further, the insert 282 may mount and/or couple using mechanical structures, such as but not limited to, the interlocking features, with or without the use of adhesives, thermal or chemical bonding, and/or other techniques to mount or couple similar or dissimilar medical grade materials.

Because the sheath can be formed by an injection molding process using molten or melted material, the shape of the sub-grooves 284 and/or other mechanical structures that facilitate mechanical coupling between two components may vary and/or accommodate many desired purposes. In some instances, the formation and/or filling of the groove 280b with the second material to form the insert 282 may cause the first material to melt, thereby causing the two materials to bond. For example, the shape of the feature 284 may include extensions that may limit the first material from separating from the second material without tearing or shearing. This may strengthen the bond, in one example, between the first and second materials. Further, the interlocking feature may ensure that the tubular portion shears at the groove 280 owing to the strength or lack thereof of the second material.

The at least one interlocking features illustrated in FIG. 3C may extend between a proximal end 232 and a distal end 234 of the tubular portion 230 and/or the introducer sheath 210. It will be understood, however, that the at least one interlocking feature may extend only part way from the distal end toward the proximal end, from the proximal end to the distal end, or at any location along the length of the tubular portion 130 and/or the sheath 210.

In addition to the use of a second material to fill the groove 280 and/or other geometric pattern, it is further contemplated that more than two materials may be utilized to form the introducer sheath and/or that other portions of the sheath may be formed from a second material. For example, a first material may be utilized to form the hub portion and one or more materials (which may include the first material) may be utilized to form the elongated portion of the sheath. Again, the selection of materials may depend on the end use of the sheath, properties of medical devices used with the sheath, and the like or any combination thereof. Although the present invention has been shown and described in accordance with specific embodiments these should not be considered limiting in any manner. For example, multiple materials may be utilized to form a unitary sheath in accordance with the present invention, wherein multiple injection molding processes are performed simultaneously or in stages to form the unitary sheath in accordance with the present invention.

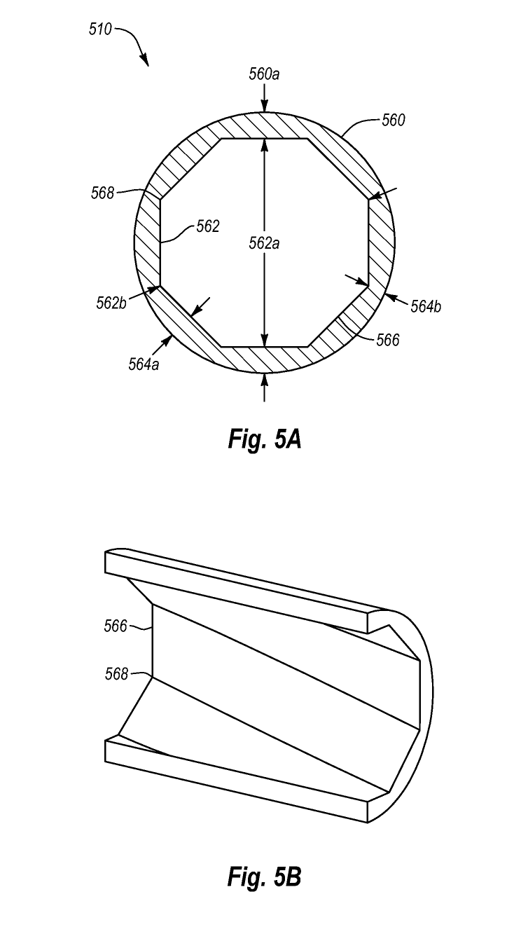

Referring generally to FIGS. 4A and 4B, there is shown a portion of an introducer sheath 410. FIG. 4A illustrates an exemplary cross-section (for example, about line 1C-1C of FIG. 1A, line 2B-2B of FIG. 2A, or line 3B-3B of FIG. 3A) of at least a portion of a single lumen introducer sheath 410. It will be understood that an irregular wall design may be incorporated into a single lumen and/or multiple lumen introducer sheath. The exemplary cross-section of the introducer sheath 410 of this embodiment may be at least partially incorporated into at least one of the introducer sheaths, 10, 110, 210 previously described above and shown in FIGS. 1A-3C or of the introducer sheaths 510, 610, 710, 810 described herein and shown in FIGS. 5A-8B. As shown in FIG. 4B, the cross-section of FIG. 4A may extend along at least a portion of the length of the introducer sheath 410.

Although the present embodiment is described with respect to a cutaway cross-section of the introducer sheath 410, the cross-section may be uniform and/or nonuniform along a length of the introducer sheath, may be uniformly and/or nonuniformly axially oriented with respect to a longitudinal axis, may be otherwise oriented, or combinations thereof.

The irregular wall surface may be formed as described herein. For example, the introducer sheath 410 may be formed by injection molding and/or other processes.

The introducer sheath 410 may include an outer surface 460 and an inner surface 462. The outer surface 460 and/or inner surface 462 may include a plurality of protrusions 466 and/or depressions 468 with a substantially uniform outer surface 462. In other embodiments, the outer surface 460 and/or the inner surface 462 may include a plurality of protrusions 466 and/or depressions 468.

The plurality of protrusions 466 and/or depressions 468 may be generally uniformly distributed about the inner circumference of the inner surface 462. In other embodiments, the plurality of protrusions 466 and/or depressions 468 may be at least partially randomly distributed about the inner or outer circumference of the inner and/or outer surface 462.

The outer wall and the inner wall of a typical elongate tubular member may define a wall thickness and an inner dimension, such as an inner diameter. In the present embodiment, the outer surface 460 and the inner surface 462 may define a plurality of wall thicknesses. For example, the outer surface 460 and the inner surface 462 may define a first wall thickness 464a between the outer surface 460 and the inner surface 462 about a protrusion 466. In another example, the outer surface 460 and the inner surface 462 may define a second wall thickness 464b between the outer surface 460 and the inner surface 462 about a depression 468.

The irregular wall design may define a plurality of inner and/or outer dimensions. For example, an inner dimension 462a, which happens to be the smallest inner diameter, may be formed through a longitudinal axis between the apexes of two protrusions 466. In another example, another inner dimension 462b, which happens to be the largest inner diameter, may be formed through a longitudinal axis between the apexes of two depressions 468. An outer dimension 460a may also be defined.

The irregular wall design formed by the protrusions 466 and depressions 468, in the present embodiment, may minimize friction between the inner surface 462 of the introducer sheath 410 and a medical device, such as a closure device delivery apparatus (not shown), to be inserted into the introducer sheath 410.

Referring generally to FIGS. 5A and 5B, there is shown a portion of an introducer sheath 510. FIG. 5A illustrates an exemplary cross-section (for example, about line 1C-1C of FIG. 1A, line 2B-2B of FIG. 2A, or line 3B-3B of FIG. 3A) of at least a portion of a single lumen introducer sheath 510. The cross-section of the introducer sheath 510 of this embodiment may be at least partially functionally similar to that of the cross-sections of the introducer sheaths, 10, 110, 210, 410, 610, 710, 810 described herein and shown in FIGS. 1A-4B and 6-8B in most respects, wherein certain features will not be described in relation to this embodiment wherein those components may function in the manner as described above and are hereby incorporated into this alternative embodiment described below. Like structures and/or components are given like reference numerals. As shown in FIG. 5B, the cross-section of FIG. 5A may extend along at least a portion of the length of the introducer sheath 510 at a non-parallel angle to the longitudinal axis of the sheath 510.

Although the present embodiment is described with respect to a cutaway cross-section of the introducer sheath 510, the cross-section may be uniform and/or nonuniformly along a length of the introducer sheath, may be uniformly and/or nonuniformly axially oriented with respect to a longitudinal axis, may be otherwise oriented, or combinations thereof.

The irregular wall surface may be formed as described herein. For example, the introducer sheath 510 may be formed by injection molding and/or other processes.

The introducer sheath 510 may include an outer surface 560 and an inner surface 562. The inner surface 562, in the present embodiment, may generally take the form of a polygonal shape, such as an octagon. Other shapes, such as a triangle, square, ellipsoid, or other shape may be used. The apexes of the inner surface 562 may define depressions 568 while the midpoint of the lines between each adjacent pair of apexes may define protrusions 566. The outer surface 560, as shown in FIG. 5A, may be substantially uniform. The plurality of protrusions 566 and/or depressions 568 are shown in FIG. 5A as generally uniformly distributed about the inner circumference of the inner surface 562.

The outer surface 560 and the inner surface 562 may define a plurality of wall thicknesses. For example, the outer surface 560 and the inner surface 562 may define a first wall thickness 564a between the outer surface 560 and the inner surface 562 about a protrusion 566 (i.e. at a midpoint of a line between two adjacent apexes). In another example, the outer surface 560 and the inner surface 562 may define a second wall thickness 564b between the outer surface 560 and the inner surface 562 about a depression 568 (i.e. at an apex).

The irregular wall design may define a plurality of inner and/or outer dimensions. For example, an inner dimension 562a, which happens to be the smallest inner diameter, may be formed through a longitudinal axis between the apexes of two protrusions 566. In another example, another inner dimension 562b, which happens to be the largest inner diameter, may be formed through a longitudinal axis between the apexes of two depressions 568. An outer dimension 560a may also be defined.

The irregular wall design formed by the protrusions 566 and depressions 568, in the present embodiment, may minimize friction between the inner surface 562 of the introducer sheath 510 and a medical device, such as a closure device delivery apparatus (not shown), to be inserted into the introducer sheath 510.

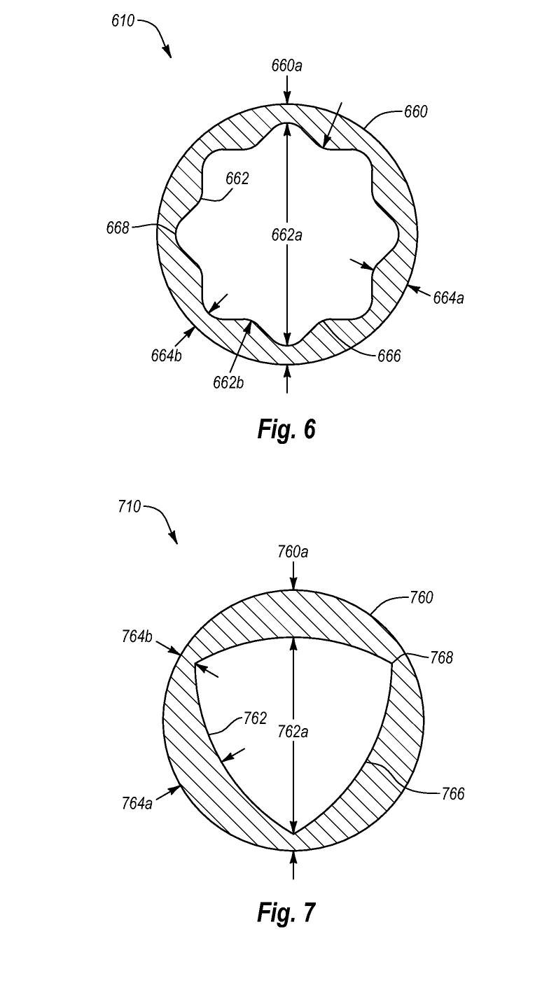

Referring to FIG. 6, there is shown a portion of an introducer sheath 610 illustrating an exemplary cross-section (for example, about line 1C-1C of FIG. 1A, line 2B-2B of FIG. 2A, or line 3B-3B of FIG. 3A) of at least a portion of a single lumen introducer sheath 610. The cross-section of the introducer sheath 610 of this embodiment may be at least partially functionally similar to that of the cross-sections of the introducer sheaths, 10, 110, 210, 410, 510, 710, 810 described herein and shown in FIGS. 1A-5B and 7A-8B in most respects, wherein certain features will not be described in relation to this embodiment wherein those components may function in the manner as described above and are hereby incorporated into this alternative embodiment described below. Like structures and/or components are given like reference numerals. The cross-section of FIG. 6 may extend along at least a portion of the length of the introducer sheath 610, such as is shown in FIGS. 4B and/or 5B and/or may vary in axial alignment with the longitudinal axis.

Although the present embodiment is described with respect to a cutaway cross-section of the introducer sheath 610, the cross-section may be uniform and/or nonuniformly along a length of the introducer sheath, may be uniformly and/or nonuniformly axially oriented with respect to a longitudinal axis, may be otherwise oriented, or combinations thereof.

The irregular wall surface may be formed as described herein. For example, the introducer sheath 610 may be formed by injection molding and/or other processes.

The introducer sheath 610 may include an outer surface 660 and an inner surface 662. The inner surface 662, in the present embodiment, may generally take the form of an octagonal shape with rounded concave portions at each apex. Other shapes, such as a triangle, square, ellipsoid, or other shape, may be used. The apexes of the concave portions of the inner surface 662 may define depressions 668 while the midpoint of the lines between each adjacent pair of apexes may define protrusions 666. The outer surface 660, as shown in FIG. 6, may be substantially uniform. The plurality of protrusions 666 and/or depressions 668 are shown in FIG. 6 as generally uniformly distributed about the inner circumference of the inner surface 662.

The outer surface 660 and the inner surface 662 may define a plurality of wall thicknesses. For example, the outer surface 660 and the inner surface 662 may define a first wall thickness 664a between the outer surface 660 and the inner surface 662 about a protrusion 666 (i.e. at a midpoint of a line between two adjacent apexes). In another example, the outer surface 660 and the inner surface 662 may define a second wall thickness 664b between the outer surface 660 and the inner surface 662 about a depression 668 (i.e. at an apex of the concave portion).

The irregular wall design may define a plurality of inner and/or outer dimensions. For example, an inner dimension 662a, which happens to be the smallest inner diameter, may be formed through a longitudinal axis between the apexes of two protrusions 666. In another example, another inner dimension 662b, which happens to be the largest inner diameter, may be formed through a longitudinal axis between the apexes of two depressions 668. An outer dimension 660a may also be defined.

The irregular wall design formed by the protrusions 666 and depressions 668, in the present embodiment, may minimize friction between the inner surface 662 of the introducer sheath 610 and a medical device, such as a closure device delivery apparatus (not shown), to be inserted into the introducer sheath 610.

FIG. 7 shows a portion of an introducer sheath 710 illustrating an exemplary cross-section (for example, about line 1C-1C of FIG. 1A, line 2B-2B of FIG. 2A, or line 3B-3B of FIG. 3A) of at least a portion of a single lumen introducer sheath 710. The cross-section of the introducer sheath 710 of this embodiment may be at least partially functionally similar to that of the cross-sections of the introducer sheaths, 10, 110, 210, 410, 510, 610, 810 described herein and shown in FIGS. 1A-6 and 8A-8B in most respects, wherein certain features will not be described in relation to this embodiment wherein those components may function in the manner as described above and are hereby incorporated into this alternative embodiment described below. Like structures and/or components are given like reference numerals. The cross-section of FIG. 7 may extend along at least a portion of the length of the introducer sheath 710, such as is shown in FIGS. 4B and/or 5B and/or may vary in axial alignment with the longitudinal axis.

Although the present embodiment is described with respect to a cutaway cross-section of the introducer sheath 710, the cross-section may be uniform and/or nonuniform along a length of the introducer sheath, may be uniformly and/or nonuniformly axially oriented with respect to a longitudinal axis, may be otherwise oriented, or combinations thereof.

The irregular wall surface may be formed as described herein. For example, the introducer sheath 710 may be formed by injection molding and/or other processes.

The introducer sheath 710 may include an outer surface 760 and an inner surface 762. The inner surface 762, in the present embodiment, may generally take the form of an Reuleaux triangle. Other shapes, such as a triangle, square, ellipsoid, or other shape may be used. The apexes of the triangle of the inner surface 762 may define depressions 768 while the midpoint of the curves between each adjacent pair of apexes may define protrusions 766. The outer surface 760, as shown in FIG. 7, may be substantially uniform. The plurality of protrusions 766 and/or depressions 768 are shown in FIG. 7 as generally uniformly distributed about the inner circumference of the inner surface 762.

The outer surface 760 and the inner surface 762 may define a plurality of wall thicknesses. For example, the outer surface 760 and the inner surface 762 may define a first wall thickness 764a between the outer surface 760 and the inner surface 762 about a protrusion 766 (i.e. at a midpoint of the curve between two adjacent apexes). In another example, the outer surface 760 and the inner surface 762 may define a second wall thickness 764b between the outer surface 760 and the inner surface 762 about a depression 768 (i.e. at an apex).

The irregular wall design may define a plurality of inner and/or outer dimensions. For example, an inner dimension 762a may be formed through a longitudinal axis between an apex of a protrusion 766 and an apex of a depression 768. An outer dimension 760a may also be defined.

The irregular wall design formed by the protrusions 766 and depressions 768, in the present embodiment, may minimize friction between the inner surface 762 of the introducer sheath 710 and a medical device, such as a closure device delivery apparatus (not shown), to be inserted into the introducer sheath 710.

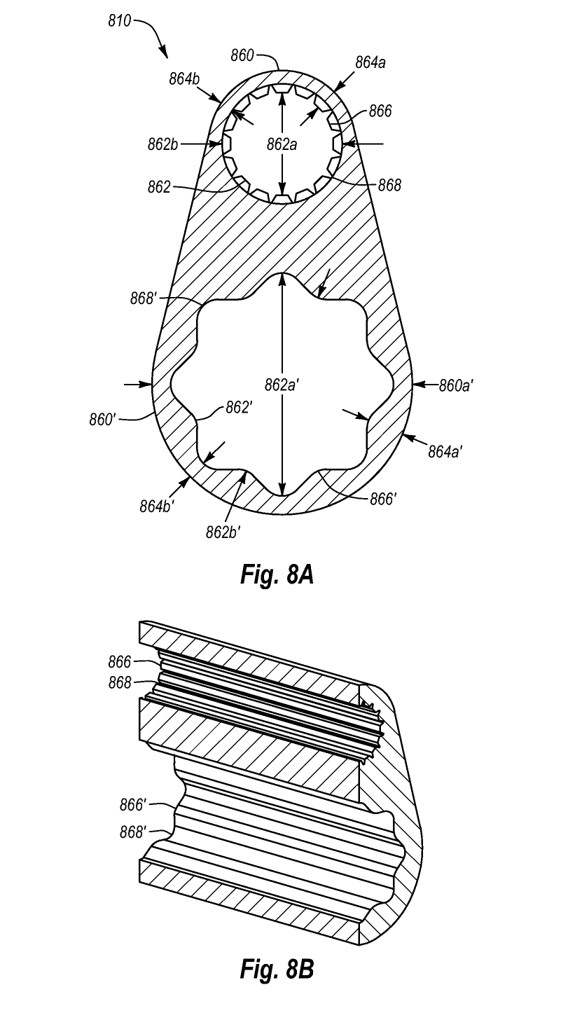

Referring generally to FIGS. 8A and 8B, FIG. 8A shows a portion of an introducer sheath 810 illustrating an exemplary cross-section (for example, about line 1C-1C of FIG. 1A, line 2B-2B of FIG. 2A, or line 3B-3B of FIG. 3A) of at least a portion of a plural lumen introducer sheath 810. The cross-section of the introducer sheath 810 of this embodiment may be at least partially functionally similar to that of the cross-sections of the introducer sheaths, 10, 110, 210, 410, 510, 610, 710 described herein and shown in FIGS. 1A-7 and 9A-8B in most respects, wherein certain features will not be described in relation to this embodiment wherein those components may function in the manner as described above and are hereby incorporated into this alternative embodiment described below. Like structures and/or components are given like reference numerals. As shown in FIG. 8B, the cross-section of FIG. 8A may extend along at least a portion of the length of the introducer sheath 810.

Although the present embodiment is described with respect to a cutaway cross-section of the introducer sheath 810, the cross-section may be uniform and/or nonuniformly along a length of the introducer sheath, may be uniformly and/or nonuniformly axially oriented with respect to a longitudinal axis, may be otherwise oriented, or combinations thereof.

The irregular wall surface may be formed as described herein. For example, the introducer sheath 810 may be formed by injection molding and/or other processes.

The introducer sheath 810 may include an outer surface 860 and a plurality of inner surfaces 862, 862′. The inner surfaces 862, 862′ may include a plurality of protrusions 866, 866′ and/or depressions 868, 868′ with a substantially uniform outer surface 862. Although the outer surface 860, as shown in FIGS. 8A and 8B, is shown without a substantially circular cross section, the outer surface 860 may be generally smooth and/or radiused. A smooth and/or radiused outer surface 860 may facilitate conforming to an atheriotomy or other insertion site. In other embodiments, the inner surfaces 862, 862′ may include a plurality of protrusions 866, 866′ and/or depressions 868, 868′ and/or may be free of protrusions 866, 866′ and/or depressions 868, 868′.

The protrusions and/or depressions in this or other embodiments may trace a linear and/or non-linear path along the length of the sheath. For example, the protrusions and/or depressions may follow a linear path along the longitudinal axis of the sheath for a portion of the sheath and then follow a non-linear or other path for another portion of the sheath.

The irregular wall design formed by the protrusions 866, 866′ and depressions 868, 868′, in the present embodiment, may minimize friction between the inner surface 862 of the introducer sheath 810 and a medical device, such as a closure device delivery apparatus (not shown), to be inserted into the introducer sheath 810 and/or between the outer surface 860 of the introducer sheath 810 and tissue which the sheath 810 may contact, such tissue near an opening in a body lumen (not shown), through which the introducer sheath 810 may be inserted.

The first inner surface 862, in the present embodiment, may generally take the form of an interior gear, similar to the shape shown in FIG. 4A. Other shapes, such as a triangle, square, ellipsoid, or other shape may be used. The outer surface 860, as shown in FIG. 8A, may be substantially uniform. The plurality of protrusions 866 and/or depressions 868 in the first interior surface 832 are shown in FIG. 8A as generally uniformly distributed about the inner circumference of the first inner surface 862.

The second inner surface 862′, in the present embodiment, may generally take the form of an octagonal shape with rounded concave portions at each apex. Other shapes, such as a triangle, square, ellipsoid, or other shape may be used. The apexes of the concave portions of the second inner surface 862′ may define depressions 868′ while the midpoint of the lines between each adjacent pair of apexes may define protrusions 866′. The plurality of protrusions 866′ and/or depressions 868′ are shown in FIG. 8A as generally uniformly distributed about the inner circumference of the first inner surface 862′.

The outer surface 860 and the inner surfaces 862, 862′ may define a plurality of wall thicknesses. For example, the outer surface 860 and the first inner surface 862 may define a first wall thickness 864a between the outer surface 860 and the inner surface 862 about a protrusion 866 and/or the outer surface 860 and the second inner surface 862′ may define a first wall thickness 864a′ between the outer surface 860 and the second inner surface 862′ about a protrusion 866′. In another example, the outer surface 860 and the first inner surface 862 may define a second wall thickness 864b between the outer surface 860 and the first inner surface 862 about a depression 868 and/or the outer surface 860 and the second inner surface 862′ may define a second wall thickness 864b′ between the outer surface 860 and the second inner surface 862′ about a depression 868′.

The irregular wall design may define a plurality of inner and/or outer dimensions. For example, a first inner dimension 862a of the first inner surface 862, which happens to be the smallest inner diameter within the first inner surface 862, may be formed through a longitudinal axis between two protrusions 866 and/or a first inner dimension 862a′ of the second inner surface 862′, which happens to be the smallest inner diameter within the second inner surface 862′, may be formed through a longitudinal axis between two protrusions 866′. In another example, a second inner dimension 862b of the first inner surface 862, which happens to be the largest inner diameter of the first inner surface 862, may be formed through a longitudinal axis between two depressions 868 and/or a second inner dimension 862b′ of the second inner surface 862′, which happens to be the largest inner diameter of the second inner surface 862′, may be formed through a longitudinal axis between two depressions 868′. An outer dimension 860a may also be defined.

The irregular wall design formed by the protrusions 866, 866′ and depressions 868, 868′, in the present embodiment, may minimize friction between the inner surfaces 862, 862′ of the introducer sheath 810 and a medical device, such as a closure device delivery apparatus (not shown), to be inserted into the introducer sheath 810.

The irregular wall design may facilitate splitting of one or more of the inner surfaces 862, 862′ and/or the outer surface 860. For example as a part of a forming process, such as injection molding, a slit and/or other weakening of the wall of one or more of the inner surfaces 862, 862′ and/or the outer surface 860, such as between the two lumens, may facilitate splitting of the introducer sheath 810.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. An introducer sheath, comprising:
a hub portion; and
an elongate tubular portion extending from said hub portion and a lumen extending from a distal end toward a proximal end of said elongate tubular portion, said lumen including a plurality of protrusions and a plurality of depressions.

2. The introducer sheath of claim 1, wherein said elongate tubular portion defines a plurality of lumens.

3. The introducer sheath of claim 2, wherein each of said plurality of lumens include a plurality of protrusions and a plurality of depressions within each lumen.

4. The introducer sheath of claim 3, wherein said plurality of protrusions define a first inner dimension within each lumen and said plurality of depressions define a second inner dimension within each lumen, said first inner dimension being smaller than said second inner dimension in each lumen.

5. The introducer sheath of claim 1, wherein said protrusions and/or depressions extend from a proximal end to a distal end.

6. The introducer sheath of claim 1, wherein said protrusions and/or depressions vary in angular orientation with respect to said longitudinal axis between a proximal end and a distal) end.

7. The introducer sheath of claim 1, wherein said plurality of protrusions and said plurality of depressions extend longitudinally through said lumen.

8. The introducer sheath of claim 1, wherein said tubular portion includes at least one weakened region and at least one stiffened region.

9. The introducer sheath of claim 1, wherein said protrusions and said depressions define a friction reducing surface configured to contact an outer surface of a medical device.

10. An introducer sheath, comprising:
a hub portion; and
an elongate tubular portion extending from said hub portion and having a first lumen and a second lumen, said first lumen including a plurality of first protrusions and a plurality of first depressions and said second lumen including a plurality of second protrusions and a plurality of second depressions.

11. The introducer sheath of claim 10, wherein said plurality of first protrusions and said plurality of first depressions differ from said plurality of second protrusions and said plurality of second depressions.

12. The introducer sheath of claim 11, wherein said plurality of protrusions and said plurality of depressions of said first inner surface are parallel from said proximal end toward said distal end.

13. An introducer sheath, comprising:
a hub portion; and
an elongate tubular portion extending from said hub portion and having a lumen, said lumen including a plurality of protrusions and a plurality of depressions, said plurality of protrusions defining a first inner dimension and said plurality of depressions defining a second inner dimension, said first inner dimension being smaller than said second inner dimension.

14. The introducer sheath of claim 13, wherein said first inner dimension is about fifty percent smaller than said second inner dimension.

15. An introducer sheath, comprising:
a hub portion; and
an elongate tubular portion extending from said hub portion and having a lumen, said lumen including a plurality of protrusions and a plurality of depressions, said plurality of protrusions defining a first wall thickness and said plurality of depressions defining a second wall thickness, said first wall thickness being larger than said second wall thickness.

16. The introducer sheath of claim 15, wherein said first wall thickness is about fifty percent larger than said second wall thickness.

17. The introducer sheath of claim 15, wherein said tubular portion includes PTFE or FEP.

18. A method for performing a medical procedure, the method comprising:
introducing a sheath into a lumen of a patient, the sheath having a first unexpanded dimension and an irregular wall surface;
inserting a first medical device having an outer dimension into the lumen through the sheath to perform a medical procedure, wherein at least a portion of a tubular member of the sheath expands to a second expanded dimension to accommodate the outer dimension of the first medical device.

19. The method of claim 18, further comprising inserting a second medical device through the sheath.

20. The method of claim 19, wherein inserting a second medical device through the sheath further comprises:
introducing a vessel closure device through the sheath; and
closing the lumen of the patient with the vessel closure device.

21. The method of claim 18, wherein the sheath comprises a tubular portion extending from the hub portion, the tubular portion comprising at least one portion deformable to increase a cross sectional area of the tubular portion.

22. The method of claim 21, wherein the at least one portion is splittable to increase a cross sectional area of the tubular portion.
